(19) United States
(12) Patent Application Publication
(10) Pub. No.: US 2010/0063395 A1
(43) Pub. Date: Mar. 11, 2010

(54) Telemedicine platform for standardized interpretation of vascular data using vascular analysis

(76) Inventors: B. Robert Mozayeni, (US); Kevin E. Crutchfield, Potomac, MD (US); Barret W. Hildebrand, Arlington, VA (US); Martin A. Cannon, Chevy Chase, MD (US); Larry J. Goldstein, Doylestown, PA (US); William Hogenauer, Doulestown, PA (US); Emad Ibrahim, Arlington, VA (US)

Correspondence Address: HOWREY LLP - East, C/O IP DOCKETING DEPARTMENT, 2941 FAIRVIEW PARK DR, SUITE 200, FALLS CHURCH, VA, 22042-2924, US

(21) Appl. No.: 12/230,800

(22) Filed: Sep. 4, 2008

Related U.S. Application Data
(63) Continuation of application No. 12/007,255, filed on Jan. 8, 2008, now abandoned.
Application No. 12/007,255 is a continuation of application No. 11/798,295, filed on May 11, 2007, now abandoned.
(60) Provisional application No. 60/799,661, filed on May 12, 2006.

(51) Int. Cl. A61B 8/00

(52) U.S. Cl. 600/453

(57) ABSTRACT

A system and method, for obtaining and analyzing vascular data and generating results, that uses vascular test data to determine the state of the vessel. The data and the determinations can be used to generate reports, render diagnoses or identifying ailments, and may do so remotely. The system includes a telemedicine server and may include a number of other modules such as work stations, review tools, data storage modules, etc. The invention allows rapid and efficient analysis of the data, and provides mechanisms for comparing patient data to know or measured normative data sets, remotely if desired, and provides more accurate and less invasive diagnoses based on vascular conditions. The invention permits remote receipt, processing and distribution of the data and diagnoses.

### CROSS-REFERENCE TO RELATED APPLICATIONS

This application is a continuation of U.S. patent application Ser. No. 12/007,255, filed on Jan. 8, 2008, entitled “TELEMEDICINE PLATFORM FOR STANDARDIZED INTERPRETATION OF VASCULAR DATA USING VASCULAR ANALYSIS”, which is a continuation of U.S. patent application Ser. No. 11/798,295, filed May 11, 2007, entitled “TELEMEDICINE PLATFORM FOR STANDARDIZED INTERPRETATION OF VASCULAR DATA USING VASCULAR ANALYSIS,” which claims the benefit of U.S. Provisional Patent Application No. 60/799,661, filed on May 12, 2006, entitled “TELEMEDICINE PLATFORM FOR STANDARDIZED TRANSCRANIAL DOPPLER INTERPRETATION USING DYNAMIC VASCULAR ANALYSIS.” The foregoing applications are hereby incorporated by reference in their entirety.

### FIELD OF THE INVENTION

The invention relates, in general, to the use of Dynamic Vascular Analysis (DVA™) (formerly described as DCA or Dynamic Cerebrovascular Analysis) and Hemodymanic Vascular Analysis (HVA™) methodologies for distinguishing among various vascular states. In particular, the invention relates to a telemedicine system, which includes both hardware and software, to automate, standardize and distribute the analysis of vascular data such as Transcranial Doppler (“TCD”) data, deploy DVA/HVA to extract more information from such data and extend neurovascular expertise world-wide for clinical and research applications. The invention further includes using such telemedicine system for assessing vascular health and the effects of treatments, risk factors and substances, including therapeutic substances, on blood vessels, especially cerebral blood vessels, but not limited thereto.

### BACKGROUND OF THE INVENTION

DVA and HVA provide methodologies of distinguishing among various vascular states. The ability to differentiate such vascular states (that may otherwise be indistinguishable until after a vascular event) is particularly applicable in many fields, one example being subarachnoid bleed from a ruptured aneurysm.

Vascular system disease processes and injury can affect the tone of a vessel or create points of blockage along the vessel (e.g., from inflammation from surrounding blood or atherosclerosis). Various methodologies exist today for assessing vascular function (more commonly referred to as endothelial function). These tests generally measure the response to a physiological stimulus such as breath holding or hyperventilation. Arterial blockages, however, are often detected by measurements of mean blood flow velocity by either Transcranial Doppler (“TCD”) ultrasound or an angiographical evaluation of the arterial segment (showing only a cross section silhouette of a vascular narrowing).

Stenosis is defined as a narrowing caused by inflammation, external compression, or arteriosclerosis within an arterial segment. Stenosis includes relative hyperemic conditions as well as vasospasm. For example, vasospasm represents a supra-physiologic stenosis given the acute development and lack of time for the vasculature to compensate. It should also be kept in mind that when there is atherosclerotic stenosis secondary to inflammatory changes at any particular point, other stenotic regions usually exist elsewhere in the vascular system (i.e., both proximate and distal to that point). The most common form of stenosis is atherosclerotic narrowing. In the coronaries and elsewhere, stenosis is assessed by a variety of methods. In the coronaries, for example, stenosis is measured primarily by angiography. As discussed above, however, angiography provides only a cross section silhouette of a vascular narrowing. As such, angiographic analysis is highly susceptible to being inaccurate (at time) due to the asymmetry of the narrowing within the artery (i.e., when the projection of view is changed, it may appear that the narrowing is either nonexistent or much smaller than would be measured physiologically).

Stenotic events and conditions resulting in significant flow alteration, including those needing therapeutic intervention, are composed of three discreet micro-physiological states depending on the three regions defined by the stenosis. The regions defined by the stenosis will be the pre-stenotic region, the stenotic region and the post-stenotic region. The three physiologic states in these regions will be a distal Perfusion-Impedance Mismatch (“PIMM”) in the pre-stenotic region, a hyperemic breakthrough at the site of stenosis in order to conserve volume and pressure of flow, and a proximal PIMM in the post stenotic region.

PIMM is defined as the imbalance of force vectors such that the impedance or resistance vector contributes more to the balance than the forward force vector. The net result of this condition is a reduction in forward flow. There may be two reasons for PIMM to occur. The first possible reason is “proximal” PIMM incurred by a drop in proximal perfusion pressure as a result of a significant stenosis. The second possible cause is a “distal” PIMM resulting from the increase in the resistance (or impedance) vector that induces the imbalance. Distal PIMM also occurs when significant small vessel disease is present. A combination of both types of PIMM can significantly inhibit forward movement of blood and when it is present in a post stenotic region it likely indicates a state of compensatory flow from other vessels.

Traditionally, neurological critical care defines two distinct types of cerebral vascular events. The first event is an ischemic flow or low flow. The second event is a vessel rupture (most commonly an aneurysm resulting from an over-dilated vessel). When a patient suffers or bleeds from an aneurysm, it typically occurs in the subarachnoid space (i.e., a subarachnoid hemorrhage). The initial response to a subarachnoid hemorrhage is a neurologic injury accompanied by loss of consciousness.

Patients surviving the initial event, however, frequently also have a secondary response to the hemorrhage. In particular, it is well documents that in the early phases of recovery, patients go into a state of hyperemia. Hyperemia is defined as a pathological increase in blood flow volume that exceeds the metabolic needs of the tissue being served by that vessel.

Another secondary response, often occurring five to ten days after the initial event, is the development of vasospasm. Vasospasm is defined as the pathologic constriction of the muscles of the vessel, causing a significant narrowing, which leads to a secondary ischemic or low flow stroke. Prevention and treatment of vasospasm (and more importantly prevention of the clinical or morbid state associated with vasospasm) primarily include hypertension and hypervolemic therapy. These therapies endeavor to increase vascular volume with fluid infusion and by raising the patient's blood pressure artificially with pharmacological agents. In the course of raising the patient's blood pressure and/or increasing the blood volume, however, it is possible to induce the state of cerebral hyperemia. Thus, treatment of one condition (vasospasm) may unintentionally induce the other (hyperemia).

As can be seen from the foregoing discussion, it is important to be able to distinguish between naturally occurring hyperemia, therapy-induced hyperemia and whether that hyperemia is actually becoming a vasospasm. The practicality of making such distinctions, however, is difficult to accomplish by traditional methodologies. For example, the current treatment modalities for vasospasm include transporting a patient to an angiography suite and performing angioplasty on the spastic lesion. Similarly, premature treatment of an apparent vasospastic condition (i.e., by hypertension and hypervolemic therapy) may actually increase a patient's risk of hyperemic swelling from the initial vascular event or cerebral edema. As such, it is critical to determine if and when a patient is transitioning from a hyperemic state to the early stages of vasospasm. Conversely, instituting hypertensive and/or hypervolemic therapy too late after the onset of vasospasm is of little or no value, as it provides no difference to the clinical outcome. In this regard, unnecessarily beginning hypertensive and/or hypervolemic therapy too far after the onset of vasospasm may be detrimental to the patient's health in view of the well known incidence of induced congestive heart failure among certain older (i.e., middle age and older) patients undergoing aggressive hypertensive and/or hypervolemic therapy.

Thus, the timing and use of hypertensive and/or hypervolemic therapy following a subarachnoid hemorrhage depends largely on being able to better define when a patient is transitioning from a hyperemic state to vasospasm. Currently, making such determinations may involve the comparison of peak systolic velocity ratios (derived from TCD ultrasound or other methodologies) of an intracranial vessel versus the extra cranial carotid artery. This comparison is referred to as the Lindegaard ratio. This type of analysis, however, is not highly accurate. Some studies have shown that the Lindegaard ratio is no better than 50% predictive for identifying the transition from hyperemia to vasospasm.

Other methodologies have been explored but have not come into widespread use for evaluating and differentiating among vascular states. One such methodology involves measuring blood pressure waves with a catheter being pulled through a point of narrowing within the corner artery. Similarly, some efforts have been directed to conducting vascular assessments using intravascular ultrasound (“IVUS”). These studies, however, have focused almost entirely on the use of the resultant ultrasound images to evaluate the physiological responses to the injection of vasodilators (e.g., adenosine) in order to calculate an anomaly defined ratio called the coronary flow volume reserve or the arterial flow volume reserve.

DVA/HVA may be used to quantitatively distinguish the transition from a hyperemic state to vasospasm (which may vary dynamically and dramatically on a day-to-day, or even moment-to-moment, basis in a neurocritical care unit). It should be further understood, however, that the physiological principals described herein may be extended and/or applied to differentiate other forms of vascular problems and vascular stenosis.

Hydrocephalus is a condition characterized by increased intracranial pressure resulting in decreased intracranial blood flow. Raised intracranial pressure puts additional external force on vessels, compressing small vessels such as terminal capillaries and/or venules. Specifically, this flow limitation affects the deeper brain structures fed by deep penetrating arteries such as those in the periventricular space. This decrease in flow characteristically results in edema formation at the ventricular horns, which is believed to be a watershed ischemic event.

Very little is known in most cases about the cause of hydrocephalus. It has been observed to affect patients with a variety of conditions including, for example, meningitis or intracranial hemorrhage (e.g., subarachnoid hemorrhage). Further, it has been speculated that it may be precipitated by certain metabolic disorders or general inflammatory states. It may also affect people, particularly the elderly, who exhibit no preexisting condition. The hydrocephalus condition often seen in the elderly is known as Normal Pressure Hydrocephalus (NPH).

Accurate diagnosis of NPH is complicated by the fact that it is characterized by the “classical symptom triad” of incontinence, dementia and unsteadiness of gait, though other symptoms are often present or more prevalent. These symptoms can often be mistakenly attributed to other causes. As a result, NPH is frequently misdiagnosed because it historically requires a high index of suspicion on the part of the treating physician. Once suspected, NPH is difficult to definitively assess and diagnose accurately. Conventionally, confirming a diagnosis of NPH may entail performing an invasive procedure, known as cisternogram, comprising injection of a radioactive tracer substance into the subdural space (i.e., the cerebrospinal fluid space) and monitoring the uptake of the tracer at particular points in the cranium using a nuclear detector at 24, 48 and 72 hour intervals after the initial injection in an effort to semi-quantitate the clearance of that radionuclide tracer.

Other methods of diagnosing hydrocephalus and NPH may include repeated lumbar puncture testing, which is the withdrawal of anywhere from 20 to 40 cc's of spinal fluid to see if a patient gains clinical improvement. The most marked improvements being in gait and mentation. Continuous pressure monitoring of the spinal fluid pressure may also be performed via an indwelling catheter. However, this methodology is performed only at those institutions having specialized critical care units dedicated to this task. Furthermore, this method entails a high risk of infection (i.e., meningitis).

While a cisternogram or other clinical study may be indicative of NPH condition, these studies alone typically do not definitively diagnose a patient with NPH because they do not sufficiently exclude other causes of the observed symptoms. The only definitive diagnostic procedure currently available entails a major invasive neurosurgical procedure. The presence of the symptoms alone, however, usually does not warrant performing such a procedure. Accordingly, it has been notoriously difficult to both accurately and quickly assess and diagnose NPH.

Finally, by the time the classic triad of symptoms appears in a patient sufficient to arouse the suspicions of the treating physician, considerable injury to the central nervous system may have already occurred. Given that the central nervous system has very little capacity for damage repair, especially in the elderly, it is highly desirable to have a system capable of being used to both preventively monitor patients before symptoms become evident and to quickly and accurately diagnose a patient once the symptoms have been expressed.

The use of the DVA/HVA methodologies described above has been uniquely applied for the diagnosis and evaluation of hydrocephalus, including NPH, both before and after surgical correction. It has been used to track the natural history and progression of the onset of NPH. It has also been used to generate a reference database useful for future diagnoses that includes a variety of intracranial pressure data such as natural history NPH data, supine data, and Trendelenberg (head down tilt of approximately 15 degrees) data.

One common shortcoming of most diagnostic systems relates to the lack of sensitivity and specificity associated with the differential diagnosis of various conditions (i.e., increased intracranial pressure and/or flow variations) that may be explain any number of physiological phenomenon. DVA/HVA enables observation of the abnormal flow characteristics in patients suffering from hydrocephalus which are especially apparent during a tilt table (Trendelenberg) test. The fundamental feature of the test is the ability to detect and observe a homogenous global increase in both the pulsatility index and flow acceleration, thus enabling discrimination between homogenous and heterogeneous affects from global intracranial events. For example, a global event could be global inflammation which would typically cause a patchy distribution when the TCD data was correlated (i.e., a heterogeneous event) or it could be a metabolic disorder affecting all vessels homogeneously without necessarily excluding any particular region. These metabolic disorders may include, for example, Fabry Disease, Diabetes or Alzheimer's Disease.

Additionally, DVA/HVA provides a means to identify critical variables that affect intracranial blood flow that in turn cause dementia. Dementia in as much as a function of deterioration of blood flow dynamics as it is due to the loss of brain tissue and deposition of pathologic substances. Accordingly, the invention provides a reliable and efficient means for diagnosing and assessing patients suffering from dementia as well as monitoring and optimizing treatments and regimens designed to combat the onset and progression of the condition.

Thus, there is a need for better diagnosis as well a decision tool to allow physicians to analyze vascular test data, such as TCD-derived data, using vascular methodologies such as DVA/HVA. Further, there is a need for a tool that provides comparisons between a patient's readings and normative data sets, as well as a system to do so.

Furthermore, the expertise to make such uses of the test data is not wide spread. As such, every location, capable of performing vascular tests on a patient, does not also have the capability to analyze, process, diagnose or otherwise use this data. Therefore, this invention, among other benefits, provides a distributed system and method that permit wide-spread or remote use of these methodologies which may achieve the benefits recited above and in the foregoing descriptions.

### SUMMARY OF THE INVENTION

The invention relates, in general, to the use of Dynamic Vascular Analysis (DVA™) and Hemodymanic Vascular Analysis (HVA™) methodologies for distinguishing among various vascular states. In particular, the invention relates to a telemedicine system, which includes both hardware and software, to automate, standardize and distribute the analysis of vascular data such as Transcranial Doppler (“TCD”) data, deploy DVA/HVA to extract more information from such data and extend neurovascular expertise world-wide for clinical and research applications. The invention further includes using such telemedicine system for assessing vascular health and the effects of treatments, risk factors and substances, including therapeutic substances, on blood vessels, especially cerebral blood vessels, but not limited thereto.

The present invention includes a system and method for analyzing vascular data, such as, but no limited to, Doppler data or TCD, with algorithms such as DVA or HVA. The data may be measured by a vascular property measuring device, such as a TCD but not limited thereto. The invention allows rapid and efficient analysis of the data, and provides mechanisms for comparing patient data to known or measured normative data sets. Further, the present invention provides more accurate and less invasive diagnoses based on vascular conditions. Additionally, the present invention also provides a methodology for differentiating among various vascular states and conditions.

In one embodiment of the present invention, such differentiation is made by a telemedicine system on TCD data deploying DVA or HVA algorithms to extract information from such TCD data. Further, the present invention may extend neurovascular expertise world-wide for clinical and research applications. The system and method of the present invention include software and hardware that distinguish between vascular states which may be used for assessing vascular health, the effects of treatments, risk factors and substances, including therapeutic substances, on blood vessels, especially cerebral blood vessels, but not limited thereto. In the present invention, a telemedicine platform enable objective, reproducible, computational processing to provide a variety of information including, but not limited to, data measures (e.g. TCD data), vascular analysis indices (e.g. DVA indices), and other parameters and hymodynamic information in a telemedicine service model across multiple instrument systems which can be supervised by a global group of experts. This extends neurovascular expertise, making it possible for facilities not associated with neurovascular centers of excellence to develop a neurovascular diagnostic capability. Further, it may help expand the use of cerebrovascular hemodynamic information in other clinical disciplines. Furthermore, other benefits of the device and method of the present invention, as well as variations on the data, data source, analysis algorithms and dissemination of the data and results, within the level of ordinary skill are contemplated here, even if not expressly stated.

### BRIEF DESCRIPTION OF THE FIGURES

FIGS. 1A, 1B and 1C illustrate the high-level components of the embodiment of the invention described below.

FIG. 2 illustrates a method according to one embodiment of the present invention.

FIGS. 3A and 3B illustrate the components of a telemedicine server according to an embodiment of the present invention;

FIGS. 4A and 4B illustrate a data review tool according to one embodiment of the present invention;

FIG. 5 illustrates a process for importing data according to one embodiment of the present invention;

FIG. 6 illustrates a process for analyzing velocimetry data according to one embodiment of the present invention;

FIGS. 7A and 7B illustrate noise removal from a velocimetry waveform according to one embodiment of the present invention;

FIG. 8 illustrates 19 vessel segments available for evaluation by a methodology such as DVA or HVA;

FIG. 9 illustrates 19 vessel segments available for evaluation;

FIG. 10 illustrates a method for reviewing velocimetry data for a patient according to one embodiment of the present invention;

FIG. 11 illustrates a method for adjusting cursor placement on a velocimetry waveform according to one embodiment of the present invention;

FIG. 12 illustrates a method for generating a report on velocimetry data for a patient according to one embodiment of the present invention; and

FIG. 13 illustrates a comparison graph comparing velocimetry data to a reference data set.

### DETAILED DESCRIPTION OF THE INVENTION

While the claims at the conclusion of the specification set forth the present invention, the following detailed description and accompanying drawings are intended to set forth a preferred embodiment for carrying out the invention. It is understood, however, that the subject matter of the present invention may be embodied in many different forms and variations known to those skilled in the art.

While the description below discusses the utilization of TCD as the data source of the invention, it should be realized that the present invention may use any data source and should not be construed as being limited to the TCD. Furthermore, the present invention may use the multivariate analysis of data from diverse sources and need not be limited to a single data source such as a TCD (e.g. use of blood pressure information).

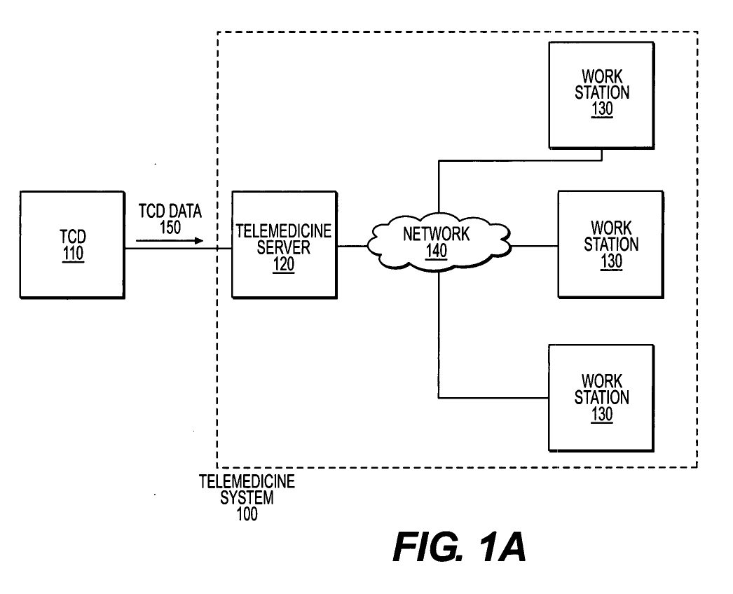

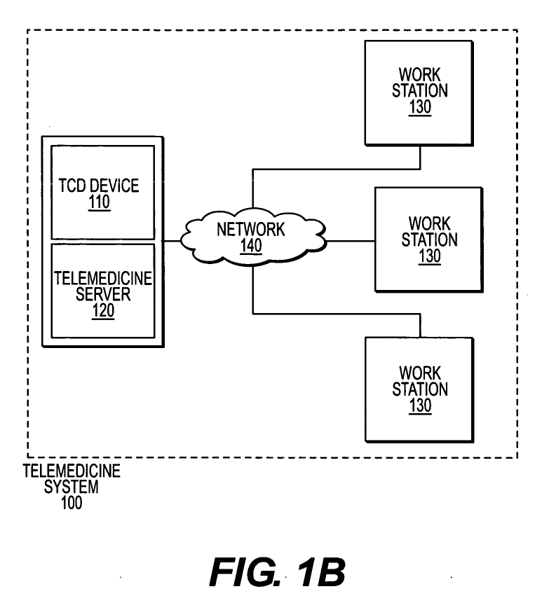

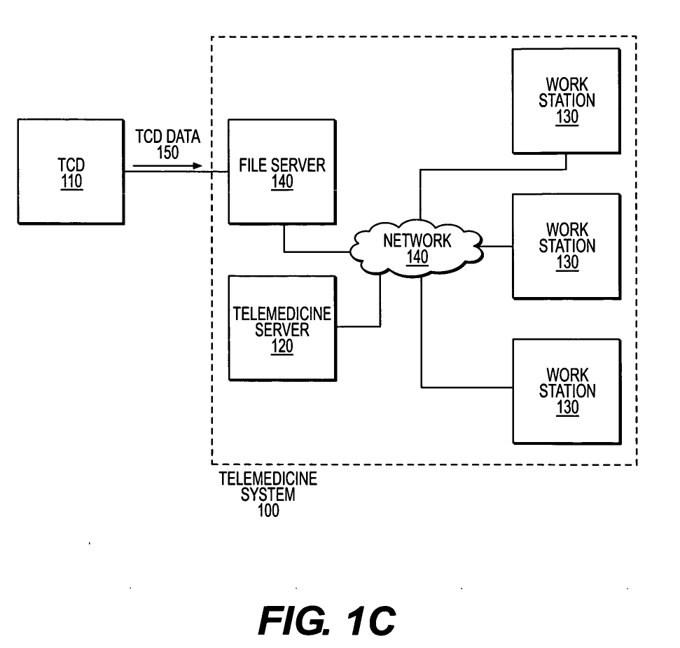

FIG. 1A illustrates a telemedicine system 100 for analyzing data, such as Transcranial Doppler (TCD) data, using, for example, decision tools for dynamic vascular analysis such as DVA or HVA, in accordance with this embodiment of the invention. In FIG. 1A, the system includes a telemedicine server 120 and a plurality of workstations 130, which may include, but are not limited to, personal computers or terminals. The workstations 130 may be located at any location in which they are capable of accessing the network 140 including, but not limited to, on-site, remote or in one or more regional centers. The server 120 receives data 150 from a device 110. The server 120 may be connected to TCD device 110 using any conventional means for connection known in the art including, but not limited to, direct connections, through an interface port, such as a parallel port or USB port, through a computer network, such as a local area network (LAN), through the Internet or wirelessly using various wireless technologies. In other embodiments, as illustrated in FIG. 1B, the device 110 may include a computer that also acts as the telemedicine server 120. In yet another embodiment, as illustrated in FIG. 1C, the device 110 may write data to a file server 140. In such an embodiment, the telemedicine server 120 is capable of reading data 150 from the file server 140.

The data 150 from device 110 may be processed on the telemedicine server 120, and users may interact with that data through the plurality of workstations 130. The workstations 130 may be connected to the server 120 through any type of conventional network 140 known to one of skill in the art. This may include, but is not limited to, a LAN or the Internet.

In operation, data 150 may flow from the device to the telemedicine server 120, where the data 150 may be processed in accordance with the methods of the present invention, as described in detail below. A user may access a review tool on a workstation 130 to review the results of the processing and may make any necessary adjustments thereto. Again, users may be located at any location including, but not limited to, on-site, remote locations or in one or more regional centers. As such, where desired, remote access is provided to the user. The adjusted data may be updated on the telemedicine server 120. After the update, the telemedicine server 120 may generate a report that may be reviewed by a user through a workstation 130. Again, where desired, the processing and storage of the data by the server and access and review by the user, as well as report generation, may be remotely performed. After generation of the report, the data and/or report may alternatively be reviewed by another user such as a physician. This again may be done remotely where desired. The physician may review the report and enter comments, interpretations or provide a diagnosis, thereby eliminating the need for the physician to dictate the comments, interpretations or diagnosis and then have that information entered on the report by a transcription service. This improves report accuracy and reduces the time required to produce a report. The physician may also electronically sign the report, after which the system will “lock” the report to prevent further modification. At this time, the physician may then send the locked report to the requesting physician. Further, the reports may be queried or viewed on-line. Any and all portions of the present invention may have remote access and any of the server, the work stations, users, physicians, data storage, report generator and any other portion of the present invention, may be located remotely to the other portions, in some cases separated by many, many miles. Access to all data in the telemedicine platform is controlled and restricted by a role-based security system. The security system prevents users from accessing any information they are not authorized to access.

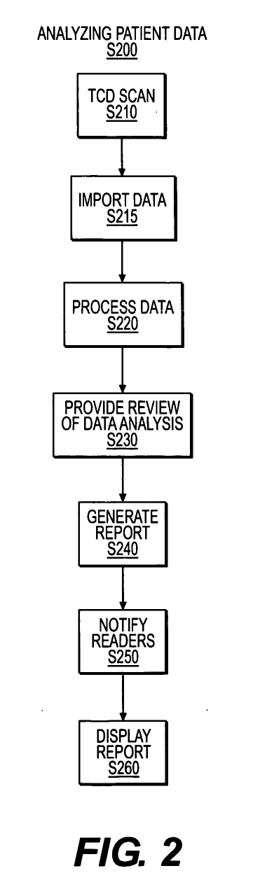

FIG. 2 illustrates a process for analyzing a patient's data according to this embodiment of the present invention. The process may begin in step S210 where the patient may be scanned by a TCD device. The data collected in step S210 may then be imported by a telemedicine server in step S215. In step S220, the telemedicine server may then process the imported data. The processing, described in detail below, may include identification of relevant features of the data for each vessel scanned. As mentioned above, in this embodiment of the present invention, this data may be Doppler data, but is not limited thereto. In step S230, a review of the result or results may be provided to enable a user to adjust the identified features. In step S240, a report may be generated that compares the patient's readings to a normative data set, for example, a reference data set. The system may optionally suggest the likelihood of certain outcomes or various diagnoses. In step S250, notification may be generated that the report is ready for review. Lastly, in step S260, the report may be displayed or printed.

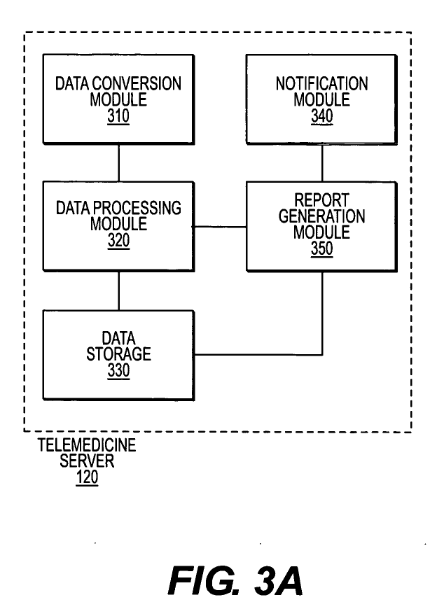

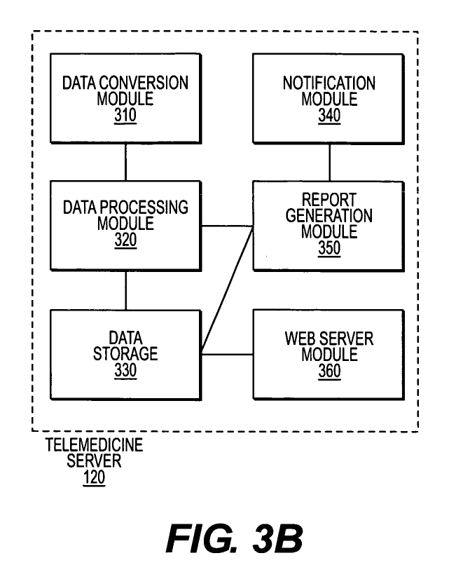

FIG. 3A illustrates one embodiment of the telemedicine server 120 that includes five modules, but is not limited thereto. The five modules shown in FIG. 3A are a data conversion module 310, a data processing module 320, a data storage module 330, a notification module 340, and a report generation module 350. While FIG. 3A illustrates a telemedicine server 120 having five specific modules, it should be realized that the server may be configured to have any number of modules, distributing the currently described functions or adding other data storage, display or processing functions. [paragraph number] The data conversion module 310 may converts the reviewed data, for example data 150 from a device 110, into a unified data format. In this regard, the device 110 could optionally output data directly into the unified data format, in which case the data conversion module 310 would leave the data unmodified. Alternatively, the data conversion module could be altogether omitted. As explained below, the data from the device 110, in this embodiment, forms a Doppler graph for each vessel scanned. The data processing module 320 takes the data formatted by the data conversion module 310 and executes algorithms to identify features of interest in the data, for example, on those Doppler graphs, and stores the results using the data storage module 330. The data storage module 330 optionally allows read and write access to this data, to processed data and/or to generated reports. The data storage module 330 may use data storage on storage space of the telemedicine server itself, storage space on another server or storage space on another device attached to or remote from the telemedicine server, storage space attached to or remote from the work stations or storage space attached to or remote from the device. Once the data produced by the data processing module has been approved, the notification module 340 may notify users that the report or reports are ready for review. The report generation module 350 produces reports/results, may allow users to review reports/results and data and may send the reports/results to the patient's physician or to a storage location.

FIG. 3B illustrates another embodiment of the telemedicine server 120 that further includes a web server module 360. The web server module 360 may provide web services that allow the device 110 to upload data, may allow users to review data, raw or processed, through a web page, and may allow users to view reports/results through a web page.

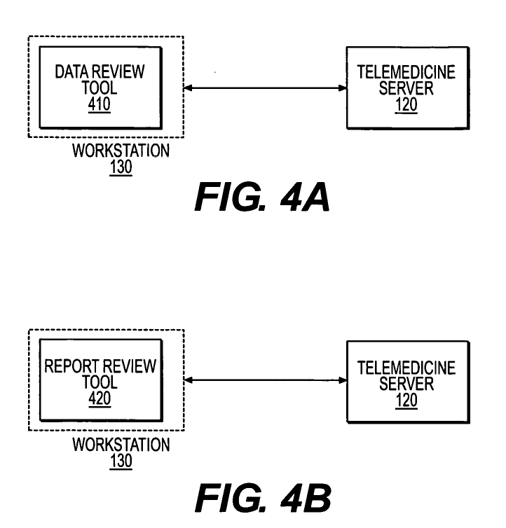

FIGS. 4A and 4B illustrate a workstation 130 which may include a data review tool 410 or a report review tool 420. These tools can take on many forms, for example, standalone applications or web-based applications, applications executing in a browser or a combination thereof. It would be apparent to one of ordinary skill in the art that any operating system, for example, Windows XP®, SunOS®, Linux or Unix®, but not limited thereto, may support the tools. One example of an implementation is in a platform-agnostic language like Java®. As illustrated in FIGS. 4A and 4B and as discussed above, workstation 130 may be connected to the telemedicine server 120 through a network.

Some examples of the data 150 provided by device 110 are listed below. This list includes, but is not limited, to the following:
Patient information—This may include information to identify the patient, for example, name, address, social security number, or a patient identification number; physical data about the patient, for example, gender, height, weight or handedness; and, medical information about the patient, like referring physician and insurance information, and other patient information
Session information—This may include the time and data of the session, for example the TCD session, a unique patient identifier, information about the person performing the procedure, and the referring physician or other session information.
Exam information—This may include a unique identification code for the exam, an accession code, the start and end times of the TCD exam, and comments of the technician or physician or other exam information.
Device information—This may include information about the TCD device, including manufacturer, model and software version or other device information.
Vascular test readings, for example vessel velocity readings other information. In the case of a Doppler reading, this may include velocimetry data, taken for each blood vessel. For each blood vessel, the data can include the fast Fourier transform data describing the velocimetry waveform. One embodiment, uses 512 time slices and 256 different sample frequencies. The data may also include an image of the waveform in a standard graphics format, such as JPEG or other graphic formats.

The format of this data can dependent upon the manufacturer of the device. Some possible formats, for example but not limited thereto, can include an XML file, a DICOM-format file, an HL 7-format file, Microsoft Access® database, a SQL-compatible database, a flat file. If necessary, the conversion of this data to the format used by the invention can be accomplished through known data mapping techniques from the format of the device into the invention's data format.

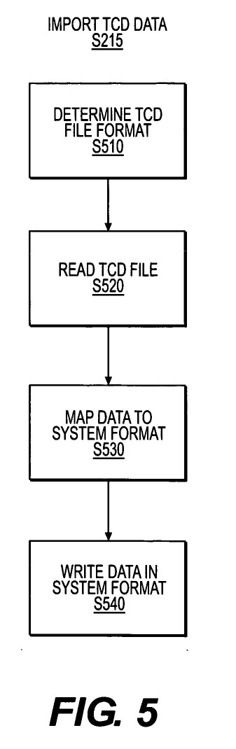

The telemedicine system 100 may have a data conversion module 310, as illustrated in FIGS. 3A and 3B. The data conversion module 310 performs the optional step S215, illustrated in FIG. 2, where data from the device 100 may be converted into the format used by the invention. FIG. 5 illustrates an example process for importing data, for example TCD data, in step S215. In step S510, the invention determines the data format. The data format in this case is determined by the manufacturer of the TCD device, and may be in any of a number of formats, including XML, a Microsoft Access®, or a relational database. The data conversion module 310 maybe configured to scan a data source for new data, or alternatively, may receive notification when data is available for conversion. Once the format is determined, data for an exam, for example from the TCD data source, may be read into memory in step S520. In step S530, the module 310 may map fields from the data read in step S520 into fields in the telemedicine data format. The mapping used in step S530 may be determined by the data format used by the device. One of ordinary skill in the art would realize how to make mapping decisions and affect this data mapping from one set of data fields to another set of data fields. In step S540, the telemedicine data may then be written to data storage. For example, the data may be written to a fixed storage, to an XML file, to storage module 330, or any suitable place without limitation.

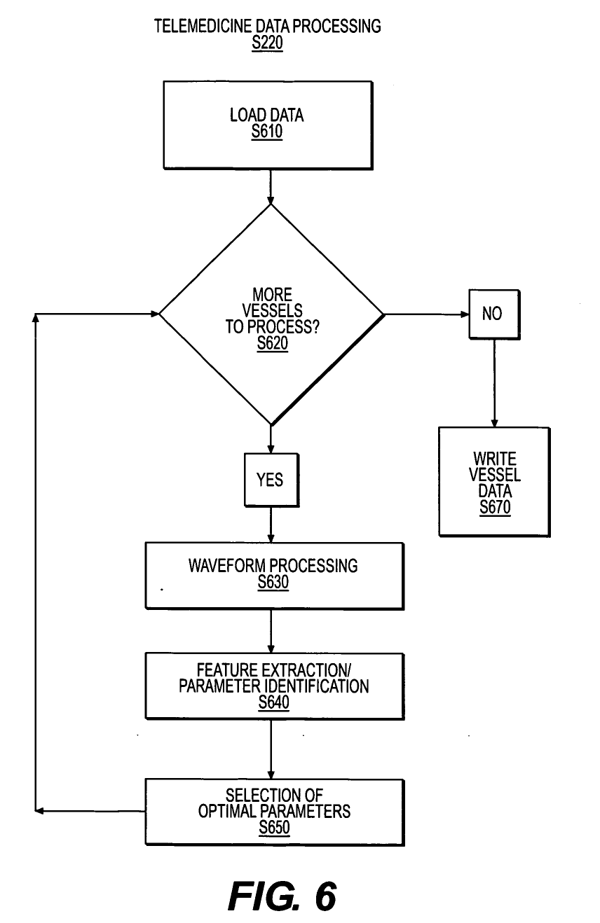

The telemedicine system 100 may include a data processing module 320, as illustrated in FIGS. 3A and 3B. The data processing module 320 performs step S220, where the data is processed to identify specific features on the data. FIG. 6 illustrates one example of a processing method for step S220 from a TCD device. The data that is processed in this example includes the velocimetry waveforms from a TCD device for each vessel. In step S610, the processing module 320 loads the data to be processed into memory from the telemedicine server. This data may have been stored by the data conversion module 310 in step S540 of the data import process S215 illustrated in FIG. 5. In step S620, if there is another vessel to process, the next vessel may be processed according to the following steps: 1) wave form processing step S630, 2) feature extraction/feature identification step S640, 3) selection of parameters of interest S650.

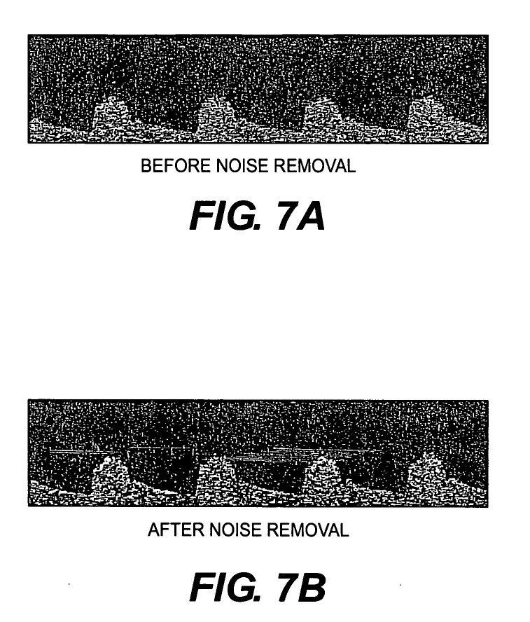

Given that ultrasound waves are echoed by objects in the body in addition to blood cells, velocimetry waveforms will often have noise from the echoes of those other objects. FIGS. 7A and 7B, illustrate data before and after the noise removal of step S60. Step S640 algorithmically identifies the relevant parameters for many useable wave forms within the Doppler data provided. Step S650 identifies the “best” wave or waves for which all identified parameters are closest to the mean parameter values for the waves within the Doppler data for that vessel. If, in step S620 there are no more vessels to process, step S670 may be performed, where the processed data, i.e., the original data plus the identified “best” waves, can be written to the telemedicine server. Other data can optionally be written to the server as well.

DVA/HVA involves the analysis of the vascular test data, for example, TCD data. As applied to evaluating and differentiating among vascular states and conditions, DVA/HVA may include TCD and/or Intravascular Ultrasound (“IVUS”) data (collectively “data”) that is collected and evaluated (via software) as a function of time and velocity. Some factors that can be measured or considered when evaluating and differentiating among vascular states are (a) a simultaneous consideration of the ultrasound data values (peak systolic velocity (PSV or Sys), end diastolic velocity (EDV or Dia), peak systolic time (PST), end diastolic time (EDT), mean flow velocity (MFV), systolic acceleration (SA), pulsatility index (PI), the natural logarithm of the SA (In SA) for each of the established 19 vessel segments within the cerebral vasculature; (b) a comparison of the data values against a reference database and/or quantifying the degree of variance from mean values; or (c) a series of indices (e.g. blood flow velocity ratios or other vascular data) that are representative of the vascular status/performance/health of each of the 19 vessel segments. Of course, the analysis need not be limited to these 19 vessel segments. Further, the list of factors above is exemplary and not exhaustive.

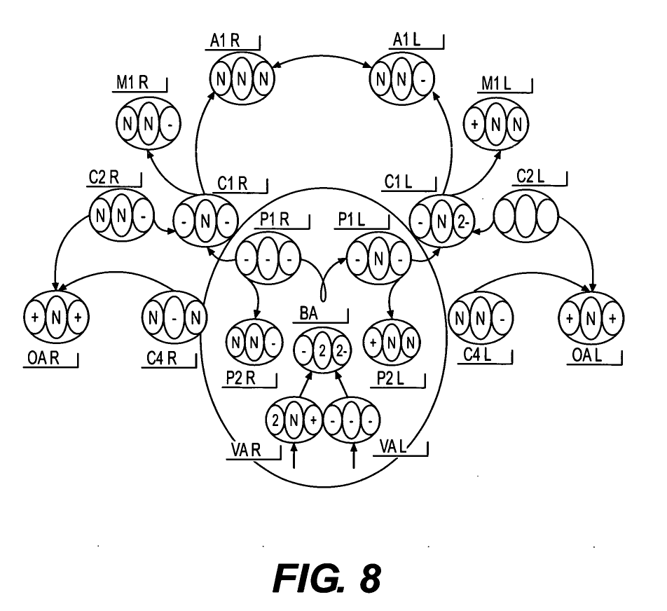

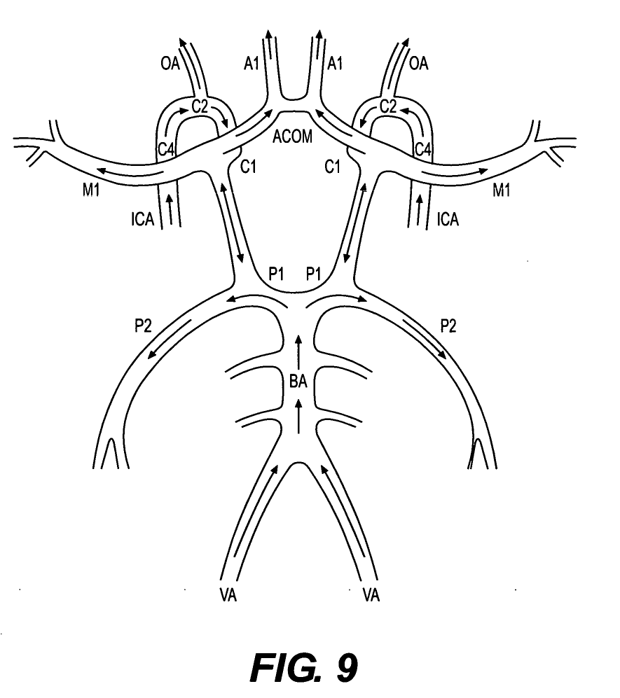

The examples of FIGS. 8 and 9 depict 19 intracranial vessel segments. The vessel segments depicted in FIGS. 8 and 9 represent the left and right vertebral artery (VA), basilar artery (BA), posterior cerebral artery/PCA t (towards)(P1), posterior cerebral artery/PCA a (away)(P2), internal carotid artery/ICA t (towards)(C1), middle cerebral artery (M1), anterior cerebral artery (A1), anterior communicating artery (ACOM), carotid siphon (towards)(C4), carotid siphon (away)(C2), and the ophthalmic artery (OA).

Peak systolic velocity (PSV) is the velocity at the identified maximum. End diastolic velocity (EDV) is the velocity at the identified minimum. The mean flow velocity (MFV) is

$$MFV = \frac{PSV - EDV}{3} + EDV$$

in approximation and more completely

$$MFV = \frac{1}{{t}_{1} - {t}_{0}} {\int }_{{t}_{0}}^{{t}_{1}} v ( t ) d t .$$

The pulsatility index (PI) is

$$PI = \frac{PSV - EDV}{MFV} .$$

The systolic acceleration (SA) is identified as the point of maximum acceleration on the velocity envelope between the end diastolic and peak systolic velocities. This value may be automatically calculated by the algorithm via known methods of calculating maxima of a data set or may be calculated via the following formula:

$$SA = \frac{PSV - EDV}{PST - EDT} .$$

The derived indices can include the dynamic work or compliance index the dynamic flow index, and the dynamic pressure index.
I. The Dynamic compliance Index (DCI or Acceleration/Mean Flow

Velocity Index (VAI)) relates to the force of flow to the mean flow velocity and describes kinetic efficiency of a segment in moving blood forward. It is given by the formula

$$DCI = \frac{InSA}{MFV} .$$

That is, the DCI is the natural logarithm of the systolic acceleration divided by the mean flow velocity.
II. The Dynamic Flow Index (DFI or Velocity/Impedance Index (VPI)) relates the mean flow velocity to the impedance (pulsatility index) and describes how capacitance volume affects flow through the conductance vessel. It is given by the formula

$$DFI = \frac{MFV}{PI} .$$

III. The Dynamic Pressure Index (DPI or Acceleration/Impedance Index (API)) relates the force of flow to impedance and describes the effect of capacitance vessel volume on the force of flow. It is given by the formula

$$DPI = \frac{InSA}{PI} .$$

That is, the DPI is the natural logarithm of the Systolic Acceleration value divided by the Pulsatility Index Value.

The basic values and derived indices may be computed based on the relevant identified features or selected parameters, in this embodiment, the maxima and minima. Thus, if cursor placements, i.e. feature identified or selected parameters are changed, the factors may be recomputed based on the new placements. As explained below, the review tool has the capability to recompute the factors dynamically as cursor placements are adjusted.

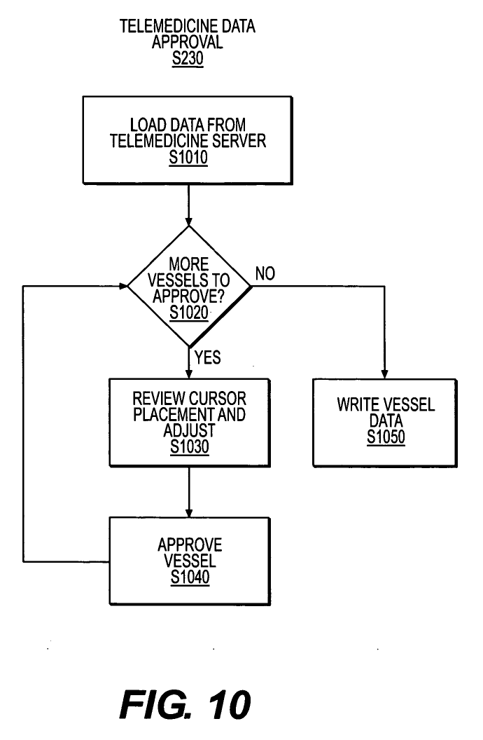

The telemedicine system 100 has a data review tool 410, as illustrated in FIG. 4A. Once the data processing step S220 has been completed, a user may perform the data review step S230, illustrated in FIG. 2, where the processed data is reviewed using the data review tool 410. One benefit of this review is to ensure that features are properly identified or the parameters appropriately selected, i.e. the features are identified/parameters are selected so that the factors computed from them are correct. FIG. 10 illustrates one method of data review S230 using the data review tool 410. In step S1010, the data review tool 410 loads vessel data from the telemedicine server. This may be performed by reading a data file from a remote server. Alternatively, other methods can be used such as requesting data from a web service. One of ordinary skill in the art would understand that other known techniques of receiving data from other devices may be used.

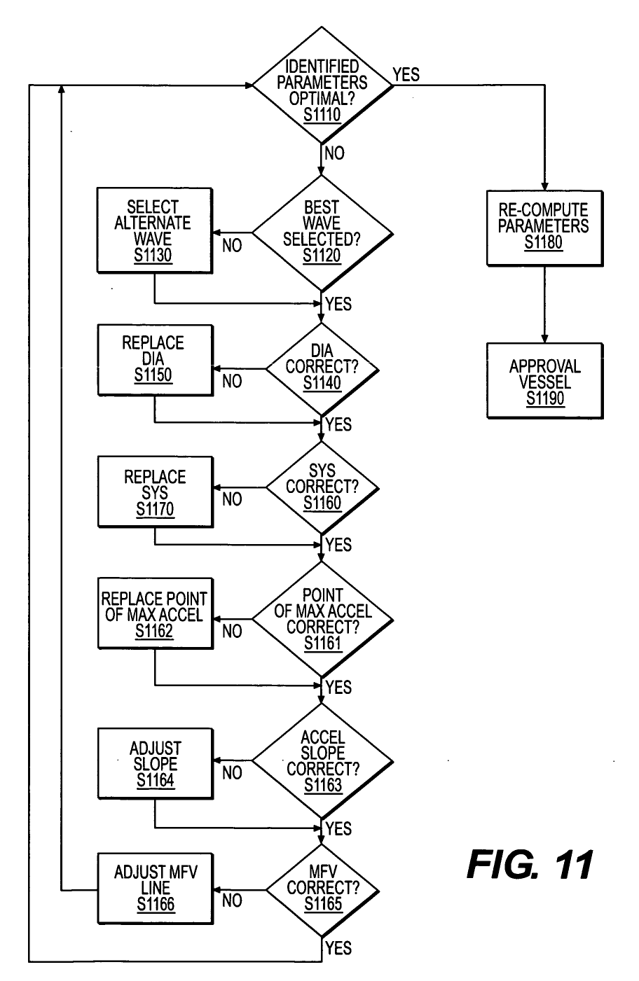

As explained above, one form of velocimetry data consists of a series of waveforms, one waveform for each vessel scanned, where features may be identified or parameters selected therefore in steps S620 to S650, as illustrated in FIG. 6. In this example, such identification or selection is done by placement of cursors to identify the features or select the parameters. The data, in this example, waveforms, may be displayed along with the cursors that identify the features. The user may then see which vessel waveforms have been reviewed or approved. The system may use various indications to distinguish reviewed or approved vessels. For example, vessel names that are reviewed or approved can be shown in color, e.g. in green. In step S1020, if vessels are remaining to be reviewed or approved, the user may select one of the unreviewed vessels and review the cursor placement in step S1030. Such a review is explained below and illustrated in FIG. 11. In FIG. 10, once the feature identification and parameter selection has been completed or approved, in this example, the placement of the cursors on a wave has been reviewed or approved, step S1030, the user reviews or approves that vessel, corresponding to the wave, in step S1040. After step S1020, step S1050 may be performed, where the updated velocimetry data may be written to the telemedicine server. In this embodiment, step S1020 concludes and step S1050 may be performed if all vessels have been reviewed or approved.

Step S230, as illustrated in FIG. 10, may include a cursor adjustment or alteration process in step S1030. Cursor adjustment here refers feature identification or parameter selection. In the embodiment described here, such identification and selection is affected by changing placement of a cursor. Nevertheless, any known method of feature identification and parameter adjustment known to those skilled in the art may be used. FIG. 11 illustrates an example of such cursor adjustment S1030 for a single vessel. In step S1110, if after looking at the waveform, the user determines that no cursor adjustment is necessary, the user can simply conclude review or approve the vessel, as in steps S1180 and S1190. Otherwise, if the user determines, in step S1120, that adjustment or alteration may be necessary, the user may perform step S1130, where the user selects and identifies the appropriate features or parameters. In this example, this selection is affected by placing the cursor on the “best” maxima and minima for each wave. In step S1140, if the peak cursor (i.e., the cursor at the maximum of the wave) is not the “best,” the reviewer performs step S1150, where he adjusts the placement of the peak cursor. In step S1160, if the valley cursor (i.e., the cursor at the minimum of the wave) is not the “best,” the reviewer performs step S1170, where he adjusts the placement of the valley cursor. It is within the level of ordinary skill in the art to repeat, vary or omit these steps or the order of performing these steps. Steps S1163 through S1166 show adjustments of cursors which are lines as well as points in this particular embodiment, related to other features of the data or waves. In step S1170, if no more adjustment is necessary, step S1180 may be performed. In step S1180 the factors for the vessel may be recalculated to reflect the new cursor placements. In step S1190 the vessel may be identified as reviewed or approved.

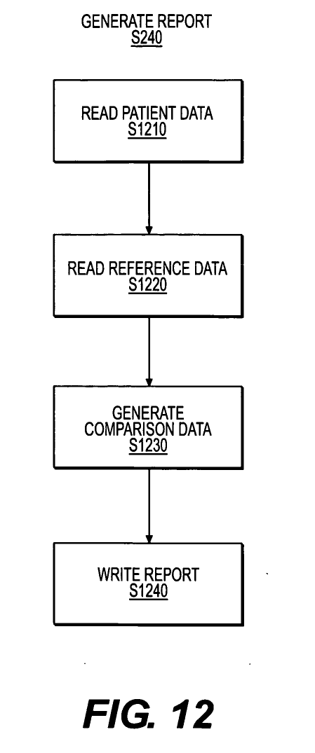

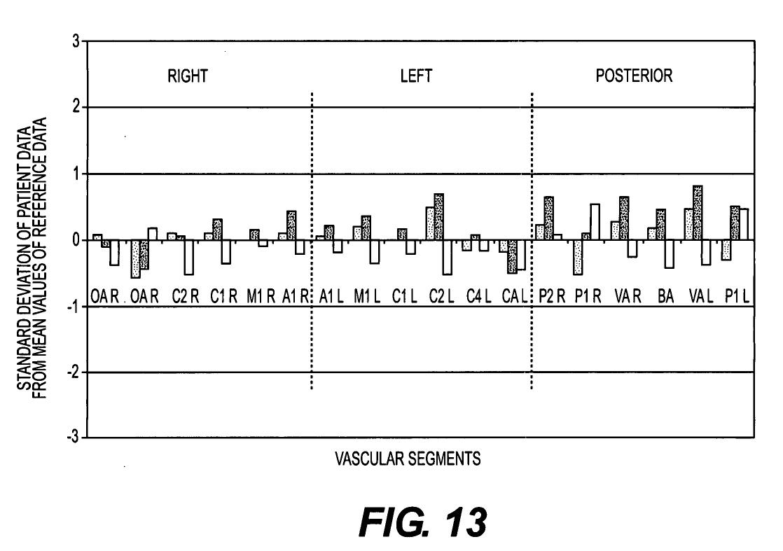

The telemedicine system 100 may have a report generation module 350, as illustrated in FIGS. 3A and 3B. The report generation module 350 may perform step S240, illustrated in FIG. 2, where the report showing the comparison between a patient's data and known reference data is generated. FIG. 12 illustrates an example process for generating a report in step S240. In step S1210, the patient data that was updated in step S230, illustrated in FIGS. 2 and 10, may be loaded into memory. In step S1220, reference patient data, e.g., data for a healthy patient of comparable physiological characteristics, is loaded into memory. In step S1230, the two data sets are compared to create a graph that may show variation of the patient's data from the reference data. An example graph is shown in FIG. 13. In step S1240, the data for the report may be written to the data storage module.

The telemedicine system 100 may have a notification module 340, as illustrated in FIGS. 3A and 3B. When step S240 is completed by the report generation module 350, the optional notification module 340 notifies readers, in step S5250, that the report is ready for display or printing, as illustrated in FIG. 2. One embodiment generates an email that is sent to an email address. Another embodiment displays a visual alert on a workstation. Other known forms of notification are also possible, including but not limited to text messages, communicating with cell phone or notification through a web page. Optionally, a webpage of reports that are ready for review may be displayed.

The telemedicine system 100 has optional report review tool 420, as illustrated in FIG. 4. After step S250 is completed by the notification module 340, readers may use the report review tool 420 to perform step S260, illustrated in FIG. 2. The report may include data relating to the patient, test device, test procedure or comparison graphs generated in step S1230, FIG. 12. The information contained in the report may be any desired information as in apparent to those skilled in the art. An example comparison graph is shown in FIG. 13. The reader may use the comparison graphs to diagnose likely conditions or to determine whether or not certain medical procedures are likely to be successful. The reader may also use the review tool to document the diagnosis or document comments by entering information into the report. After the reader has concluded entering information into the report, the reader may indicate so by any suitable method. For example, the reader may electronically sign the report. After the conclusion of the entry or “signing” of the report, the report may be “locked” to prevent further modification. The locked reports may be sent back to the server or to one of the storage devices or to other users. Further, readers can optionally query or review reports on-line. Further still, access to the reports, data, or the entire system altogether can be optionally role-based and restricted to certain users or optionally have various levels of security or require various levels of authentication of the user. The readers can be located any where including on-site, remote or in one or more regional centers.

While the foregoing explanations are made to better illustrate and describe the invention, they are not intended to limit the scope of the claims. The scope of the invention is to be defined by the claims appended hereto, and by their equivalents, and all equivalent structures, acts and configurations known to those skilled in the art are contemplated herein.

What is claimed is:

1. A system for obtaining and analyzing vascular test data, the system comprising:
a vascular test data source capable of providing source data;
a receiving unit coupled to the data source and capable of receiving said source data and capable of processing said source data to generate processed data;
at least one station coupled to the receiving unit, the station permitting review of said processed data;
wherein the receiving unit includes a processor to affect said processing;
wherein said processing includes executing vascular analysis algorithms on said source data to identify features of interest in said data and thereby identify vascular states; and
wherein at least one of the test data source, the receiving unit and the at least one station is located remote from the other of said at least one of the test data source, the receiving unit and the at least one station.

2. The system of claim 1, wherein the data source is a Doppler test machine.

3. The system of claim 1, wherein the stations permit reselection of said identified features.

4. The system of claim 1, wherein the system is configured to generate a report of the source data or the processed data or both.

5. The system of claim 4, wherein the generated report is locked against further modification.

6. The system of claim 1, wherein the remotely located one of said the test data source, the receiving unit and the at least one station communicates with the system through a communication network.

7. The system of claim 6, wherein the communication network is the internet.

8. A vascular data processing unit for processing vascular data, said unit comprising:
a data processing module for processing received vascular data;
a data storage module for storing vascular data;
a report generation module for generating reports based on said vascular data;
wherein the data processing module executes vascular analysis algorithms on said data and identifies features of interest in said data, said features capable of identifying vascular states; and
wherein the processing unit is capable of being coupled to a vascular testing device; and
wherein the processing unit is capable of communicating said vascular data with a station located remotely there from.

9. The receiving unit of claim 8, in combination with said station.

10. A method for obtaining and analyzing vascular test data comprising: receiving vascular test data, from a data source, in a receiving unit;
executing one or more vascular analysis algorithms on said data to identify features of interest in said data;
accepting adjustment or reselection of said features of interest;
generating a report form said accepted adjustments or reselections;
wherein at least one of said executing, accepting and generating occurs remotely from the other of said executing, accepting and generating.

11. The method of claim 10, wherein the data source is a vascular testing device.

12. The method of claim 10, wherein the data source is a Doppler test machine.

13. The method of claim 10, wherein the data source is a Transcranial Doppler test machine.

14. The method of claim 10, wherein said adjustment or reselection occurs on at least one station in communication with said receiving unit.

15. The method of claim 14, wherein said at least one station is remote from said receiving unit.

16. The method of claim 14, wherein said communication occurs through the internet.

17. The method of claim 10, wherein the receiving unit is coupled to data storage.

18. The method of claim 10, further including conversion of said source data from one format to another.

19. The method of claim 10, further including generating a report of the source data or the processed data or both.

20. The method of claim 19, further including locking said report after said generation.

21. The method of claim 20, wherein said locking occurs after receipt of input triggering said locking.

22. The method of claim 21, wherein said input is received from a remote location.

23. The method of claim 10, wherein the remotely occurring one of said executing, accepting and generating is communicated to the other of said executing, accepting and generating through a communication network.

24. The method of claim 23, wherein said communication network is the internet.
